(19) United States
(12) Patent Application Publication
(10) Pub. No.: US 2005/0276791 A1
(43) Pub. Date: Dec. 15, 2005

(54) Multi-layer polymer scaffolds

(75) Inventors: Derek J. Hansford, Columbus, OH (US); Sun Yang, Columbus, OH (US); Nick Ferrell, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University

(21) Appl. No.: 11/063,523

(22) Filed: Feb. 22, 2005

Related U.S. Application Data
(60) Provisional application No. 60/546,439, filed on Feb. 20, 2004.

(51) Int. Cl. A61K 45/00; C12N 5/08

(52) U.S. Cl. 424/93.7; 424/423; 435/366

(57) ABSTRACT

Three-dimensional single or multilayer polymer scaffolds for use in tissue engineering and other applications are provided. These scaffolds typically include at least two layers of biodegradable polymer of similar thickness, wherein each layer of polymer further includes a plurality of substantially uniform structural features having predetermined geometries, and wherein each layer of polymer is attached to the other layers of polymer to form predefined spatial relationships between the structural features of each layer.

### CROSS-REFERENCE TO RELATED APPLICATIONS

This patent application claims the benefit of U.S. Provisional Patent Application Ser. No. 60/546,439 filed on Feb. 20, 2004 and entitled “Multi-Layer Micromolding of Precisely Machined Polymers,” the disclosure of which is incorporated by reference as if fully rewritten herein.

### STATEMENT REGARDING FEDERALLY FUNDED RESEARCH

This invention was not made by an agency of the United States Government nor under contract with an agency of the United States Government.

### TECHNICAL FIELD OF THE INVENTION

This invention relates in general to devices and method for use in tissue engineering and other fields, and in particular to polymer scaffolds having certain specifically designed structural features and other predetermined geometric characteristics. BACKGROUND OF THE INVENTION [0004] Tissue engineering is known by those skilled in the art to be a practical and promising approach to addressing the scarcity of donor organs available for allograft treatment. Support devices or substrates having three-dimensional structures have been utilized in the past to support cell growth in wound healing and tissue regeneration. Such devices have also been shown to have certain effects on cell behaviors, such as proliferation, migration and differentiation. Research on contact guidance indicates that surface patterns affect cell motion and orient cell location. These effects are enhanced when cells grow on precisely designed features which encourage the development of the correct intracellular structures. Thus, in some circumstances, cell growth, migration, proliferation, and differentiation are regulated by surface topographical factors such as ridges, islands, wells, or similar structures.

Certain geometrical morphologies on surfaces are known to improve cellular adhesion, proliferation, and functionality and cells typically respond most strongly to feature dimensions (e.g., 1-10 μm) that are a fraction of their minimum dimension. Photolithography and microfabrication techniques are known to be effective means by which to fabricate surface features at micro or nano scales. However, precision control of microfeature geometry remains a difficult problem to overcome. Three-dimensional scaffolds with feature sizes of 50-100 μm, or even several hundreds of microns are typically limited in usefulness for the study of individual cell behaviors and are not capable of precise tissue replacement. Thus, there is a need for a method for fabricating polymer scaffolds having precisely designed and controlled structural features at the micro- or nano-scale.

### SUMMARY OF THE INVENTION

The present invention relates to multilayer thermoplastic polymer scaffolds, which are useful for tissue engineering applications such as wound healing and tissue regeneration, and other applications. This invention includes both the three-dimensional polymer scaffolds and an exemplary method for fabricating the scaffolds.

In accordance with one aspect of the present invention, three-dimensional single or multilayer polymer scaffolds are provided. In an exemplary embodiment, each scaffold includes at least two film-like layers of polymer, wherein each layer of polymer is about 1 μm to 10 μm thick and wherein each layer of polymer further includes a plurality of substantially uniform structural features, the dimensions of which are measurable in micrometers (microns) or nanometers. These micro or nano features are engineered by photolithography or other means to exhibit specifically designed or “predetermined” geometries and characteristics. Additionally, each layer of polymer is attached to the other layers of polymer to form predefined spatial relationships between the structural features of each layer.

In accordance with another aspect of the present invention, a method for fabricating single or multilayer thermoplastic polymer scaffolds is provided. In an exemplary embodiment, this method includes the steps of fabricating a master template having predetermined geometric characteristics; coating the surface (raised and recessed areas) of the master template with a solution of a first polymer and allowing the first polymer solution to solidify; removing the solidified polymer from the master template to form a polymer stamp, wherein the polymer stamp further comprises a plurality of geometric surface features, and wherein these features further comprise a plurality of raised and recessed areas; coating the polymer stamp with a solution of a second polymer; removing the excess second polymer solution from the surface of the polymer stamp such that the second polymer solution remains substantially in the recessed areas of the stamp; using pressure to transfer the second polymer solution to a substrate and allowing the second polymer solution to solidify to form a single-layer polymer scaffold on the substrate; and detaching the polymer scaffold from the substrate.

Additional features and aspects of the present invention will become apparent to those of ordinary skill in the art upon reading and understanding the following detailed description of the exemplary embodiments. As will be appreciated, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIG. 1 is an optical micrograph of the SU-8 master 45-45 μm grid pattern on a silicon wafer.

FIG.2 is an optical micrograph of the polydimethylsiloxane (PDMS) stamp with the 45-45 μm grid pattern.

FIG.3 is a schematic diagram of the primary steps of the method of the present invention: (a) spin coat uniform polycaprolactone (PCL) layer on pre-patterned PDMS mold; (b) remove PCL from raised portions of mold; (c) micro-transfer molding of PCL scaffold layer onto glass slide; and (d) repeat process to build up multiple layers of pre patterned PCL scaffold.

FIG. 4. is a series optical micrographs of the one-layer grid-patterned scaffolds.

FIG. 5 is a series of optical micrographs of the two-layer grid-patterned scaffolds.

FIG. 6 is a series of scanning electron microscopy (SEM) micrographs of two-layer grid-patterned scaffolds: (a) top view; (b) tilted view.

FIG. 7 is a series of SEM micrographs of four-layer grid-patterned scaffolds: (a) low magnification, showing uniformity of pattern; (b) higher magnification, top view, showing 30° rotation between layer alignments; (c) higher magnification, tilted view, showing connections between layers; (d) high magnification, tilted view, showing continuous weld between two layers.

FIG. 8 is a series of fluorescent microscopy images of cell growth in the scaffolds: (a) on a one-layer grid-patterned scaffold; (b) on a two-layer grid-patterned scaffold.

FIG. 9 is a series of SEM micrographs of cell growth in the scaffolds: (a) on a one-layer grid-patterned scaffold; (b) on a five-layer grid-patterned scaffold.

### DETAILED DESCRIPTION OF THE INVENTION

The present invention relates to single and multilayer thermoplastic polymer scaffolds, which are useful for tissue engineering applications such as wound healing and tissue regeneration, as well as other applications such as, for example, photonic crystals and meta-materials for antennas. This invention includes both the three-dimensional polymer scaffolds and an exemplary method for fabricating the scaffolds.

In an exemplary embodiment, each scaffold includes at least two layers of polymer, wherein each layer of polymer is about 1 μm to 10 μm thick, wherein each layer of polymer further includes a plurality of substantially uniform structural features having predetermined shapes and other geometric characteristics, and wherein each layer of polymer is attached to the other layers of polymer to form predefined spatial relationships between the structural features of each layer. Each layer of the polymer scaffolds includes specifically or precisely controlled, i.e., created or fabricated, geometric structures including, for example, ridges and grids. Square, rectangular, triangular, circular, oval, hexagonal and trapezoidal subunits are also possible. These geometric structures are engineered to be microscale features, i.e., measurable in microns (about 1-10 μm, for example) or nanoscale features, i.e., measurable in nanometers. Thus, a significant range of sizes is possible with the method of the present invention. The scaffolds are “film-like” and offer distinct advantages due to their ability to maximize the surface contact and interactions between grafts and tissue. High porosity and interconnected pores are useful inherent properties of the multilayer scaffolds. In an exemplary embodiment, biodegradable polycaprolactone (PCL) is used as the structural material for these scaffolds.

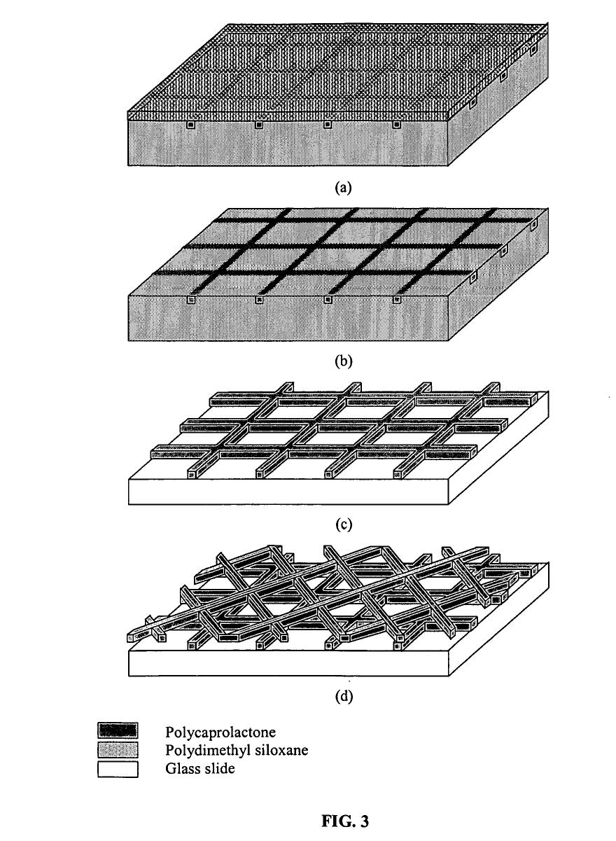

As partially shown in FIG. 3, an exemplary embodiment of the multi-layer micro-molding method of the present invention is useful for the fabrication of three-dimensional (“3-D”) scaffolds having a precise 5 μm line-45 μm space grid pattern for use in tissue engineering applications or other applications. This method permits discrete control of the size, shape, and spacing of support structures within the scaffolds and includes the basic steps of fabricating a master template having predetermined or predefined geometric characteristics; coating the master template with a solution of a first polymer and allowing the first polymer solution to solidify; removing the solidified polymer from the master template to form a polymer stamp, wherein the polymer stamp further comprises a plurality of specifically designed surface features, and wherein the features further comprise a plurality of raised and recessed areas; coating the polymer stamp with a solution of a second polymer; removing the excess second polymer solution from the surface of the polymer stamp such that the second polymer solution remains substantially in the recessed areas of the stamp; transferring the second polymer solution to a substrate and allowing the second polymer solution to solidify to form a single-layer polymer scaffold on the substrate; and detaching the polymer scaffold from the substrate. Multiple molding steps create layers having independent features that allow precise alignment between the various layers. Additionally, a high surface to volume ratio reduces the amount of polymer required, thereby reducing degradation byproducts.

In the exemplary embodiments, soft lithography techniques are utilized for fabricating polydimethylsiloxane (PDMS) stamps with the desired grid pattern. Appropriate heating and stamping techniques are used for micromolding the thermoplastic polymer and the multiple layers of the scaffold are precisely aligned and welded. In an exemplary embodiment, a microfabricated seven-layer scaffold provides a vertical height of 35 μm.

In exemplary embodiments that include single layer scaffolds, uniform features are achieved over the entire stamp with a diameter of about 2.5 cm. Scanning electron microscopy (SEM) may be used to characterize the scaffold structures. The high porosity (e.g., 81% by design) and the abundant interconnections are inherent advantages of the scaffolds and are important to understand certain fundamental cell behaviors. Static cell cultures grown on the scaffolds indicated that cell membranes and cytoskeletons are shaped by the scaffold structures and cell adhesion and location are regulated by the scaffold structures. Initial cell growth testing results were done on both of the 2-D and 3-D grid-patterned scaffolds, and demonstrated enhanced cell adhesion and spreading.

The step-by-step process of this method is relatively simple and straight-forward. PCL can be replaced by any other kind of thermoplastic, biodegradable material; therefore, the applications of the technique are very broad. Photolithography techniques are capable of providing various designs for the scaffold structures. The flexibility is important for practical uses of the scaffolds. In addition, multiple inexpensive PDMS stamps can be generated from one master, which improves the economical aspects of this invention. Multiple layers of the 3-D scaffolds are easy to align and different scaffold geometries are useful for cell studies and other research. Layers were successfully aligned with specific angles so that a number of various sized pores were produced. Also, applying different pattern designs can alternate the structure of the scaffolds. For example, the sizes of the grid, or even the patterns of different layers can be altered. Thus many shapes can be derived such as triangles, hexagons, or more complicated geometries. In this sense, the total geometries of the scaffold can be controlled during fabrication. By designing the scaffold with computer aided design (CAD), it is possible to match the tissue growth in vivo with cell differentiation in vitro.

Having generally described this invention, a further understanding can be obtained by reference to certain specific examples detailed below, which are provided for purposes of illustration only and are not intended to be all inclusive or limiting unless otherwise specified.

### EXAMPLE

### Preparation of Polymer Solution

Polycaprolactone (PCL) pellets (Aldrich Chemical, Wis.) with an average Mₙ of ca. 80,000 (GPC) and a melting point of 60° C. (DSC) were used for the fabrication of scaffolds. A PCL solution was developed to fully wet the polydimethylsiloxane (PDMS) mold. At room temperature, PCL was dissolved in tetrahydrofuran (THF) (Mallinckrodt Baker, Inc. NJ) in a 1:3 ratio by weight. The mixture was left overnight until all of the polymer pellets were fully dissolved and the final solution was clear and transparent. Next, Dimethyl Sulfoxide (DMSO) (Sigma-Aldrich, Wis.) was added into the polymer solution in a weight ratio of 3:2. The mixture was stirred thoroughly and resulted in a clear solution. Incorporation of DMSO allowed the polymer solution to wet the hydrophobic PDMS surface and thus be delivered into the small grid features within the PDMS. The double solvent solution was employed since PCL was not soluble with DMSO, so THF was used to first dissolve the polymer. From the above process, the overall PCL solution concentration was 10 wt %, with a ratio of PCL:THF:DMSO=1:3:6 (weight ratio). This concentration was found to be more effective than 2%, 5%, 15%, and 20% (all by weight) for scaffold fabrication, so it was used throughout the following fabrication processes.

### Microfabrication of Grid-Patterned Masters

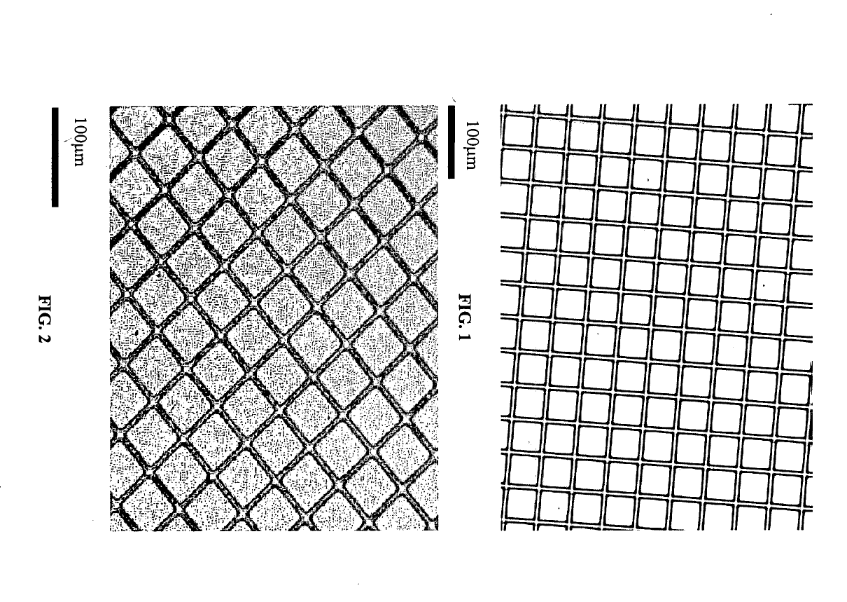

Five-micron thick NANO™ SU-8 negative photoresist (MicroChem Co., Mass.) was spun onto a 100 mm (100) silicon wafer (WaferTec, Inc.). For all experiments, a chrome on glass mask consisting of 5 μm open lines separated by 45 μm dark spaces (5-45 line mask) was used. The sizes of these lines were selected based on the expected cellular attachment to the 5 μm features that could be produced and to allow proper transport to and from cells grown on the scaffolds. The 5-45 μm line mask was exposed twice to obtain a silicon/SU8 master with 5 μm wide lines with 45 μm square spaces (5-45 μm grid pattern). After the first exposure, the mask was turned 90 degrees and exposed again, producing two groups of lines that were perpendicular to each other and resulted in the 5-45 μm grid pattern. Optical microscopy showed that the doubly-exposed intersection portions of the SU8 were not noticeably overexposed (i.e. no noticeable rounding of the corners) and the two-step exposure had no negative effect on the features of the master. The pattern was subsequently developed according to manufacturer instructions and dried under a gentle stream of nitrogen. The precise pattern of 5-45 μm grid pattern was successfully fabricated and the microscopy result is shown in FIG. 1.

### Soft Lithography for PDMS Stamps

PDMS prepolymer was prepared by mixing the translucent base with curing agent (Dow Corning, Mich.) by a 10:1 w/w ratio. After thorough mixing, the prepolymer was poured on the top of the master. The whole system was then placed in a vacuum desiccator to remove air bubbles in the PDMS generated during mixing. After it was degassed, the PDMS prepolymer was left overnight at room temperature until it became a solid elastomer. The PDMS was peeled off the silicon and cut into a round stamp with a 2.5 cm diameter. The PDMS stamps were inspected under an optical microscope to ensure that the 45-45 μm pattern was successfully transferred, as shown in FIG. 2. In other embodiments of this invention, other thermoplastic polymers are substituted for PDMS for use in making the stamps.

### Multilayer Micromolding Method

An exemplary embodiment of the multi-layer micro-molding method is shown in FIG. 3. The first step of the multi-layer micro-molding method was the transfer molding of the base layer of the scaffold. The PCL solution was dropped onto the PDMS stamp and allowed to spread for 20 seconds then spun over the surface of the stamp using a spin coater (Specialty Coating Systems, Inc., NH). The solution was spun at 1000 rpm for of 60 seconds. After spin coating, the PCL solution was evenly coated across the surface of the stamp. The polymer and the stamp were left under a fume hood overnight to remove the solvents. Two digital hot plates (Extech Instruments, MA) were set at 85° C. and 70° C., respectively. Stamps coated with the PCL and the precleaned glass slides were heated on the 85° C. hotplate. When the polymer was completely melted, the PDMS stamp with the PCL was stamped onto the glass slide. When the stamp was peeled off the slide, excess polymer on the raised features of the stamp surface (an array of 45 μm squares with 5 μm spacing) was removed and polymer was only retained in the stamp grooves. This procedure was repeated several times to ensure the removal of the entire excess polymer layer.

The treated stamp with the PCL in the grooves was moved to the 70° C. hot plate and left there for several minutes until the system had a uniform temperature and the PCL became soft. A new precleaned glass slide was heated to 70° C. and served as the substrate. The stamp was pressed with the PCL side on the glass slide at 70° C. and 35 psi force was manually applied by reading the forcedial (Wagner, Md.) on a stamping press. The stamp and the glass substrate were cooled to room temperature (20° C.) in ambient air. When cooled below its T_{g}, the PCL became stiff and shrank slightly. Thus the stamp was easily peeled off the glass slide, leaving the polymer grids on the glass slide. This served as the base layer of the scaffold.

After the base layer was made, two hot plates were adjusted to 35° C. and 70° C. respectively. Each layer of the scaffold was prepared within the features of an individual PDMS stamp according to the above-described process to remove the excess polymer on the raised features described for the base layer above. The PCL-loaded stamps were heated to 70° C. until the PCL was softened. Concurrently, the base layer of the scaffold was heated to 35° C. for 30 seconds such that the polymer was slightly softened for the purpose of welding between layers. Three marks were labeled on the glass slide in advance, with a 30-degree angle between every two marks. Then the stamp was pressed with 11 psi onto the base layer, shifting the orientation 30 degrees from the base layer orientation. After pressing, the stamp and the slide were cooled to room temperature (20° C.). The stamp was peeled off the slide, leaving the second layer attached to the base layer. The same procedure was repeated to achieve multiple layers of the scaffold. For subsequent layers of the scaffold, care was taken to soften only the top PCL layer on the glass slide. This was accomplished by holding the polymer about 2 mm above the hot plate with an approximate temperature of 35° C. Using this technique, good welding between layers was achieved and the previous layers retained their rigidity, maintaining the 3-D structure of the scaffolds. The resulting scaffolds were characterized with optical microscopy and scanning electron microscopy (SEM) respectively.

### Cell Culture in the Scaffolds

Human osteosarcoma cells (HOS) were subcultured as a monolayer suspension in 75 cm² polystyrene flasks. The cells were cultured in Minimum Essential Eagle's Medium (Dulbecco's, Wis.), with 1% antibody and 10% (v/v) fetal bovine serum (FBS) for several generations. The cell culture atmosphere was maintained as a mixture of 95% air and 5% CO₂. A seeding density of 10⁵/cm² was used for the cell culture in the polymer scaffolds. Adherent cells were enzymatically released using 0.05% trypsin and counted using a hemocytometer. Scaffolds on the glass slides were sterilized under UV light overnight and placed in the 6-well culture plate. The cells were seeded into the scaffolds and incubated at 37° C.

### Fluorescent Staining

After 24 hours of culture, 1 ml of 10 μM Cell Tracker green (CTG) was added to each well and the cells were incubated for an hour. The cells were fixed with ethanol and placed in a freezer at −20° C. for 30 minutes. Finally the cells along with the scaffolds were mounted with Fluoromount-G and ready for fluorescent microscopy (CompuCyte, MA).

### Sample Preparation for SEM Characterization

For SEM observation, cells on the scaffolds were fixed with 2% glutaraldehyde in 0.1M phosphate buffer with 0.1M sucrose overnight at 4° C. Next the cells were permanently fixed with 1% osmium tetroxide (OsO₄) (1 hr, 20° C.). The cells were dehydrated in graded ethanol (50%, 70%, 80%, 95%, 100%), mixtures of absolute ethanol (E) and hexamethyldisilazane (HMDS, H) (E:H=3:1, 1:1, 1:3, volume ratio), and subsequently dehydrated with HMDS only. The samples were dried in a fume hood and stored in a vacuum dessicator. Finally, the samples were coated with a sputtered gold layer (Techniques Hummer II) and examined in the SEM (Hitachi S-3000H) at an acceleration voltage of 5 KeV.

Using the exemplary method described above, a seven-layer grid scaffold was manually fabricated in about one hour. The resulting scaffold could be detached from the glass slide and conformed to surfaces of various shapes without delamination. The surface area of the scaffold was approximately 5 cm² with a thickness of 5 μm for each layer. By manually stretching the scaffold, it was determined that it had excellent mechanical properties and the ability to withstand a large tensile stress. The pattern features were stable and did not change for months under normal ambient air conditions. Due to the repetitive and precise features of the grid-patterned scaffold as well as the vertical sidewall features of each layer, the porosity of the whole scaffold was calculated from the 2D pattern to be about 81%.

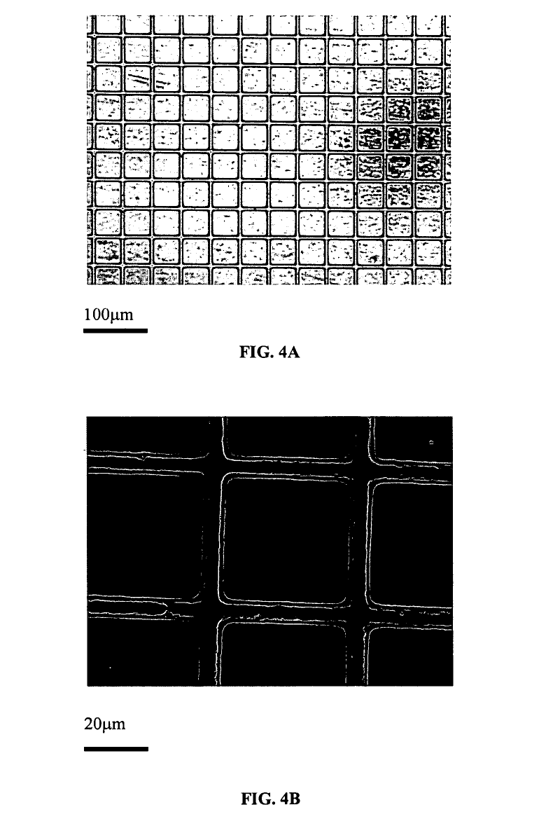

Optical microscopy images of a single grid-patterned layer are shown in FIGS. 4A-B. FIG. 4A shows that the PCL layer on the glass slide was substantially uniform and the 45-45 μm grid pattern was well preserved. The whole layer was clean and uniform, with little or no excess polymer found on the layer. The higher magnification image of FIG. 4B shows that the polymer grid had the edge and space with an approximate ratio of 1:9. The edges and corners of the grids were sharp and straight without significant deformations. Slight irregularities were seen due to the crystalline regions of the PCL. Manual tensile detection showed that the grids would not break even when extended to ten times their original length. Instead the grid structure was deformed and resulted in irregular grids.

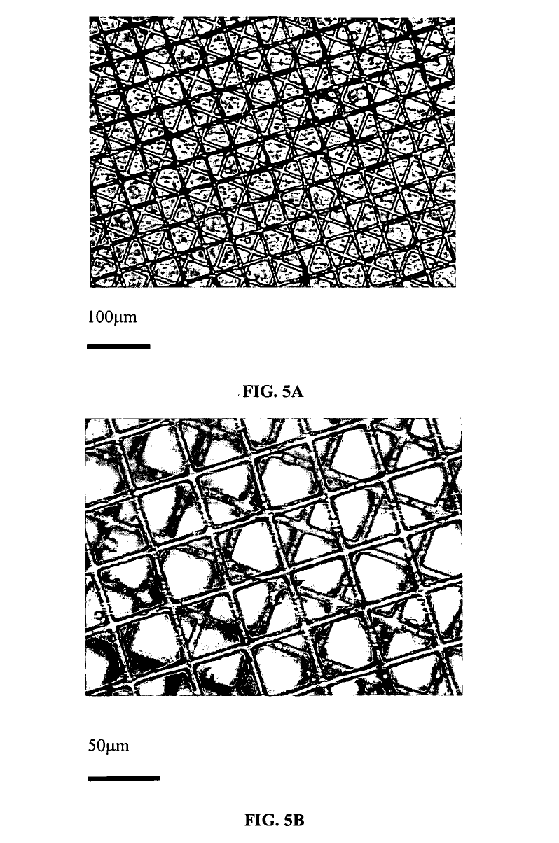

Optical microscopy images of multilayer grid-patterned scaffolds are shown in FIG. 5A-B. FIG. 5A shows that the scaffolds had substantially uniform and precise features over the entire stamp area, as with the single layer results. The scaffold was clean and there was little or no excess polymer left in the spaces of the grids. The 4-45 μm grid pattern was well preserved in each layer without any significant deformation. Different layers were successfully aligned with specific angles so that a number of various sized pores were produced. Generally, the pore sizes differed from zero to the maximum of 2025 μm² (45 μm×45 μm). In addition, the depth of the pores varied from about 5 μm to 35 μm, based on the number of the layers (5 μm/layer with up to 7 layers). The pores throughout the scaffold were highly interconnected as designed, which can impart significant benefits for cell growth for tissue engineering applications. Under higher magnification optical microscopy (FIG. 5B), it is evident that the edges of each layer were sharp and substantially straight without any significant deformation. The corners of each layer were preserved well so that there were many precise lines, corners, and steps provided for cell adhesion and attachment.

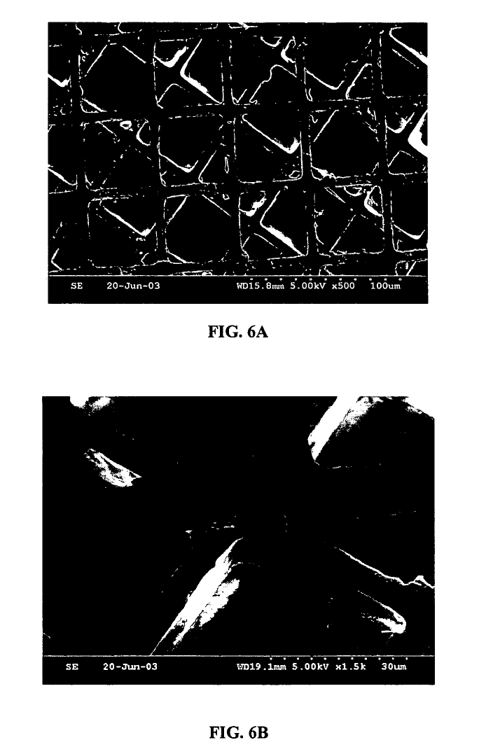

SEM images of the scaffolds fabricated with the exemplary multi-layer micro-molding method of this invention are shown in FIGS. 6A-B. FIG. 6A demonstrates the scaffolds with two grid-patterned layers and it displays that the 5-45 μm grid pattern was well preserved during the multi-layer micro-molding process. FIG. 6B shows that the two layers were built up to form a 3-D structure and that there was no appreciable bending or twisting of the base layer or collapsing of the upper layer. It is evident from the images that the 45-45 μm grid pattern was precisely preserved for every layer of the scaffold. The good welding and melting at the contact surface between the two layers enhanced the mechanical rigidity of the scaffold structure. This property is an important result of the multi-layer micro-molding method.

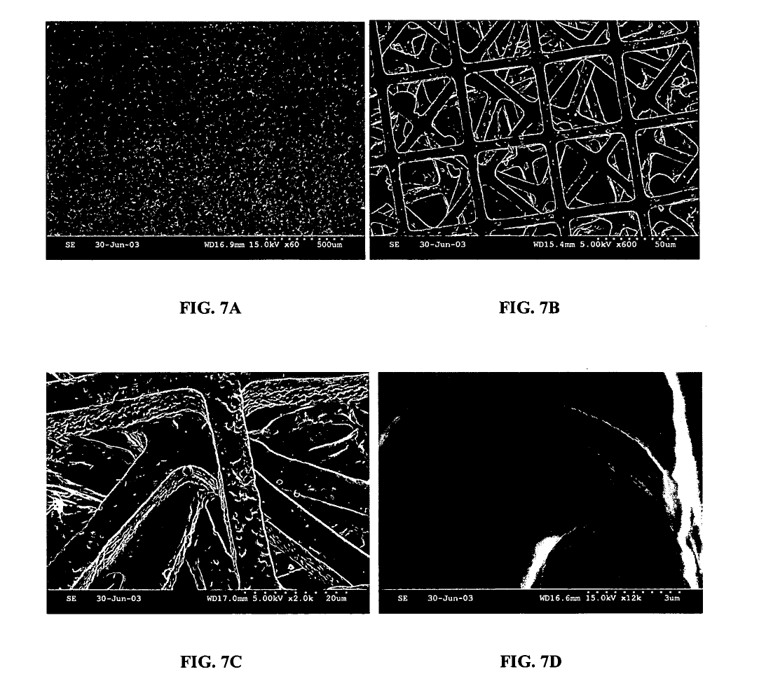

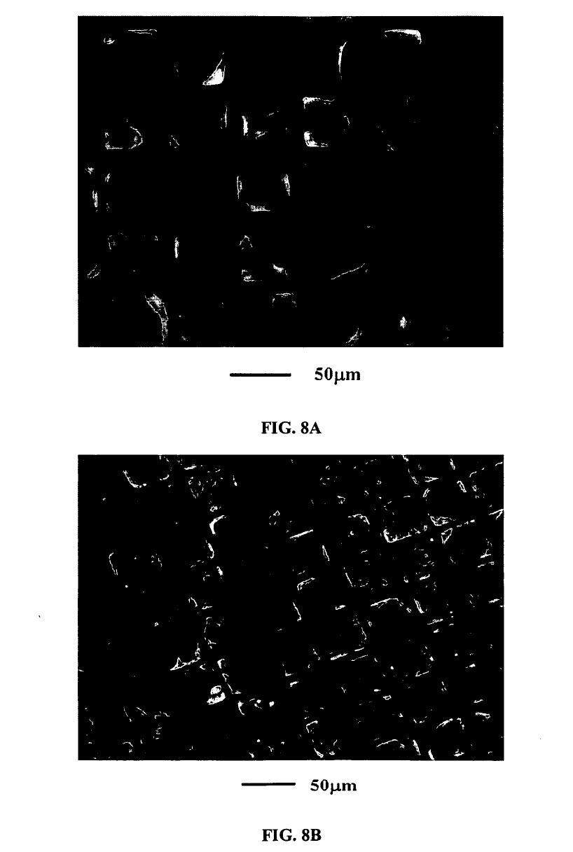

SEM images of four-layer scaffolds are shown in FIGS. 7A-D. FIG. 7A shows that the scaffold had substantially uniform and continuous profiles across the whole stamp (2.5 cm diameter). The whole surface of the scaffold was also clean and smooth. A close-up is shown in FIG. 7B. The four layers had proper alignment with a 30° alignment shift between the adjacent layers. Overall the 45-45 μm grid pattern was well preserved in all of the layers. The edges of the grid were sharp and straight without any deformations. There was no significant collapsing or twisting or bending in each layer. The interconnections due to welding, perpendicular to the direction of the layers, were clearly demonstrated.

The tilted view of the edges (see FIG. 7C) shows that both the stacking and alignment were performed precisely. The edges were smooth and straight, which demonstrates good mechanical rigidity of the layers. Upper layers were well supported without any collapsing or bending. Thus the whole structure of the scaffold was 3-D while maintaining a highly interconnected pore structure. This property is important in tissue engineering because it provides enough porosity for cell growth and transport of nutrients and wastes. It is clear that the width of the edges was approximately 5 μm and the space between every two edges was approximately 45 μm square. This demonstrates that the multi-layer micro-molding method preserved the features of the original PDMS stamps very well and the process was successful in producing a prototype scaffold. FIG. 7D shows the contacting portion of two layers, showing the excellent welding between the layers and the continuous connection. There was no appreciable twisting or bending for each layer. It demonstrates that the multiple layers were not just simply stacked together, but were welded together, which enhanced structural rigidity and made the scaffold easy to handle. This feature is also very useful in studying cell-substrate interactions.

Fluorescent microscopy results of cell culture on the glass slide and flat PCL surface without any patterns showed that cells grew on the glass randomly and cells had difficulty attaching and growing on the flat PCL. FIG. 8A shows the fluorescent microscopy result of the cell culture on the one-layer grid-patterned scaffold. It is evident that cells grew evenly across the entire grid area. Also, cells were found to attach preferentially at the corners or along the edges of the grids. In addition, cell morphology was influenced by the scaffold geometry. This demonstrates that the grid pattern had the desired effects on regulating cell adhesion and location. A fluorescent microscopy image of the cell culture results on the two-layer grid-patterned scaffolds is shown in FIG. 8B. It is evident from these images that cells grew evenly over the grid-patterned scaffolds. Cells grew preferentially at the corners of the grids and cell morphology in general conformed to the shape of the corners. The image demonstrates that the 3-D grid-patterned scaffolds can spatially regulate cell growth.

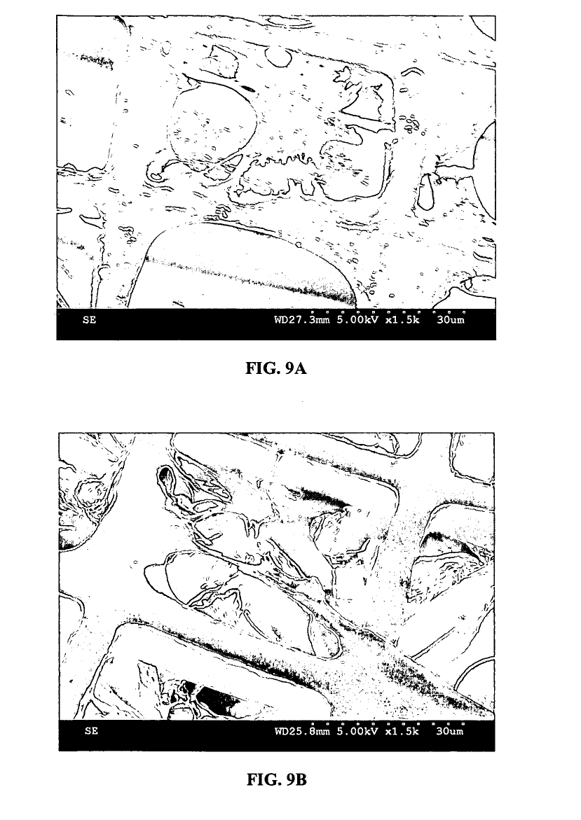

SEM characterization demonstrated several key results from the 3-D static cell culture inside the scaffolds. It is shown in FIG. 9A that cells spread their membranes over the edges of the grids, and the attachment of the membranes to the PCL grids was strong enough to suspend the whole cell body above the substrate. Similar results were observed across the entire scaffold. In FIG. 9B, cells were found growing into the multilayer scaffold structures and adhering to the underlying layers or even to the glass substrate. Clearly, cells grew into the interconnections of the scaffold and connected between the different layers even in a static culture. This result is very significant in terms of the scaffolds of the present invention being used for tissue regeneration and wound healing.

The three-dimensional polymer scaffolds of the present invention provide numerous advantages over prior art devices and methods. First, these scaffolds provide highly porous architectures and interconnections without “dead-ends” so that cells are easily exposed to adequate nutrients and oxygen. Second, the repetitive multilayer structure is uniformly fabricated across the entire stamp area, which in the exemplary embodiment is about 5 cm². Third, various sizes of tissue patches are desirable for tissue engineering applications, and the exemplary method provides a means for fabricating such devices separately. Fourth, the layers of the scaffold are sufficiently interconnected and adjacent layers have good welding across the entire scaffolds. This avoids the delamination of the allograft, which is a common problem with other systems. Finally the multilayer grid structure is beneficial for cell adhesion and spreading, which is essential for cells to survive and proliferate. The scaffolds hold potential for studying single cell behaviors, especially cell differentiation. Cells actively respond to the surface features and grow in various ways and, in general, cells make significant use of the space inside the scaffold.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the applicant's general inventive concept.

1. A polymer scaffold, comprising:
(a) at least one layer of polymer; and
(b) wherein the at least one layer of polymer further comprises a plurality of substantially uniform structural features having predetermined geometries.

2. The polymer scaffold of claim 1, wherein the dimensions of the structural features are measurable in microns.

3. The polymer scaffold of claim 1, wherein the dimensions of the structural features are measurable in nanometers.

4. The polymer scaffold of claim 1, further comprising additional polymer layers and wherein each layer of polymer is attached to the other layers of polymer at a predetermined angle to form predefined spatial relationships between the structural features of each layer.

5. The polymer scaffold of claim 4, wherein each layer of polymer is about 1 μm to 10 μm thick.

6. The polymer scaffold of claim 1, wherein the polymer further comprises a biodegradable polymer.

7. The polymer scaffold of claim 1, wherein the plurality of substantially uniform structural features further comprises square, rectangular, triangular, circular, oval, hexagonal or trapezoidal subunits.

8. A polymer scaffold, comprising:
(a) at least two layers of polymer;
(b) wherein each layer of polymer further comprises a plurality of substantially uniform structural features having predetermined geometries; and
(c) wherein each layer of polymer is attached to the other layers of polymer at a predetermined angle to form predefined spatial relationships between the structural features of each layer.

9. The polymer scaffold of claim 8, wherein the dimensions of the structural features are measurable in microns.

10. The polymer scaffold of claim 8, wherein the dimensions of the structural features are measurable in nanometers.

11. The polymer scaffold of claim 8, wherein each layer of polymer is about 1 μm to 10 μm thick.

12. The polymer scaffold of claim 8, wherein the polymer further comprises a biodegradable polymer.

13. The polymer scaffold of claim 8, wherein the polymer further comprises polycaprolactone.

14. The polymer scaffold of claim 8, wherein the plurality of substantially uniform structural features further comprises square, rectangular, triangular, circular, oval, hexagonal or trapezoidal subunits.

15. The polymer scaffold of claim 6, further comprising living biological cells seeded onto the scaffold.

16. A method for making a polymer scaffold:
(a) fabricating a master template having predetermined geometric characteristics;
(b) coating the master template with a solution of a first polymer and allowing the first polymer solution to solidify;
(c) removing the solidified polymer from the master template to form a polymer stamp, wherein the polymer stamp further comprises a plurality of structural features corresponding to the geometric characteristics of the master template; and wherein the structural features further comprise a plurality of recessed areas;
(d) coating the polymer stamp with a solution of a second polymer;
(e) removing any excess second polymer solution from the surface of the polymer stamp such that the second polymer solution remains substantially in the recessed areas of the stamp;
(f) transferring the second polymer solution to a substrate and allowing the second polymer solution to solidify to form a single-layer polymer scaffold on the substrate; and
(g) detaching the polymer scaffold from the substrate.

17. The method of claim 16, further comprising the step of attaching additional layers of polymer scaffolds to the first polymer layer prior to removing the scaffold from the substrate.

18. The method of claim 17, wherein each additional layer of polymer scaffolding is attached to the layer beneath it at a predetermined angle to form a predefined spatial relationship between the structural features of each layer.

19. The method of claim 16, further comprising the step of seeding the polymer scaffold with living biological cells.

20. The method of claim 16, wherein the master template further comprises a silicon substrate coated with a negative acting photoresist material.

21. The method of claim 16, wherein the predetermined geometric characteristics and structural features of the master template are fabricated by photolithography means.

22. The method of claim 16, wherein the predetermined geometric characteristics further comprise a grid pattern.

23. The method of claim 22, wherein the grid pattern further comprises substantially uniform square, rectangular, triangular, circular, oval, hexagonal, or trapezoidal subunits.

24. The method of claim 16, wherein the first polymer is a thermoplastic polymer.

25. The method of claim 16, wherein the first polymer is polydimethylsiloxane.

26. The method of claim 16, wherein the stamp has a surface area of about 2.5 cm².

27. The method of claim 16, wherein the second polymer is a biodegradable polymer.

28. The method of claim 16, wherein the second polymer is polycaprolactone.

29. The method of claim 16, wherein the substrate is a glass slide.

30. A method for promoting cell proliferation, comprising:
(a) preparing a sample of living cells;
(b) seeding the living cells on an artificial three-dimensional substrate; wherein the artificial three-dimensional substrate comprises:
(i) at least two layers of polymer;
(ii) wherein each layer of polymer further comprises a plurality of substantially uniform structural features having predetermined geometries; and
(iii) wherein each layer of polymer is attached to the other layers of polymer at a predetermined angle to form predefined spatial relationships between the structural features of each layer.

31. The polymer scaffold of claim 30, wherein the dimensions of the structural features are measurable in micrometers.

32. The polymer scaffold of claim 30, wherein the dimensions of the structural features are measurable in nanometers.
